(19) United States
(12) Patent Application Publication
(10) Pub. No.: US 2008/0262576 A1
(43) Pub. Date: Oct. 23, 2008

(54) METHOD, SYSTEM, AND APPARATUS FOR SOMATIC TREATMENT

(76) Inventors: Alan Austin Creamer, Carlsbad, CA (US); Gerald McLaughlin, Rutland, VT (US)

Correspondence Address: Richman and Associates, PO Box 3333, La Jolla, CA, 92038-3333, US

(21) Appl. No.: 12/107,063

(22) Filed: Apr. 21, 2008

Related U.S. Application Data
(60) Provisional application No. 60/925,549, filed on Apr. 20, 2007.

(51) Int. Cl. A61N 5/06; A61B 18/18

(52) U.S. Cl. 607/88; 607/93; 607/100; 604/20; 606/13

(57) ABSTRACT

Embodiments of somatic cell treatment are described generally herein. Other embodiments may be described and claimed.

### CROSS REFERENCE TO RELATED APPLICATIONS

The present application claims the benefit of priority to U.S. Provisional Application No. 60/925,549, entitled “System for Somatic Treatment,” filed on Apr. 20, 2007, the entirety of which is incorporated by reference.

### TECHNICAL FIELD

Various embodiments described herein relate generally to treating somatic tissue, including systems, and methods used in treating somatic tissue.

### BACKGROUND INFORMATION

It may be desirable to treat somatic tissue, the present invention provides such treatment.

### BRIEF DESCRIPTION OF THE DRAWINGS

FIG. 1A is a simplified diagram of a somatic treatment system according to various embodiments.

FIG. 1B is a simplified, cross section diagram of a somatic treatment system according to various embodiments.

FIG. 1C is a simplified, cross section diagram of layers of another somatic treatment system according to various embodiments.

FIG. 1D is a simplified, cross section diagram of layers of another somatic treatment system according to various embodiments.

FIG. 2 is a simplified diagram of a somatic treatment system according to various embodiments.

FIG. 3A is a simplified diagram of another somatic treatment system according to various embodiments.

FIG. 3B is a simplified diagram of a further somatic treatment system according to various embodiments.

FIG. 4 is a simplified diagram of a somatic treatment system according to various embodiments.

FIGS. 5A-6 are diagrams of signals that may be applied to one or more somatic treatment systems according to various embodiments.

FIGS. 7A-7C are flow diagrams illustrating somatic treatment system processing algorithms according to various embodiments.

FIG. 8 is a block diagram of an article according to various embodiments.

### DETAILED DESCRIPTION

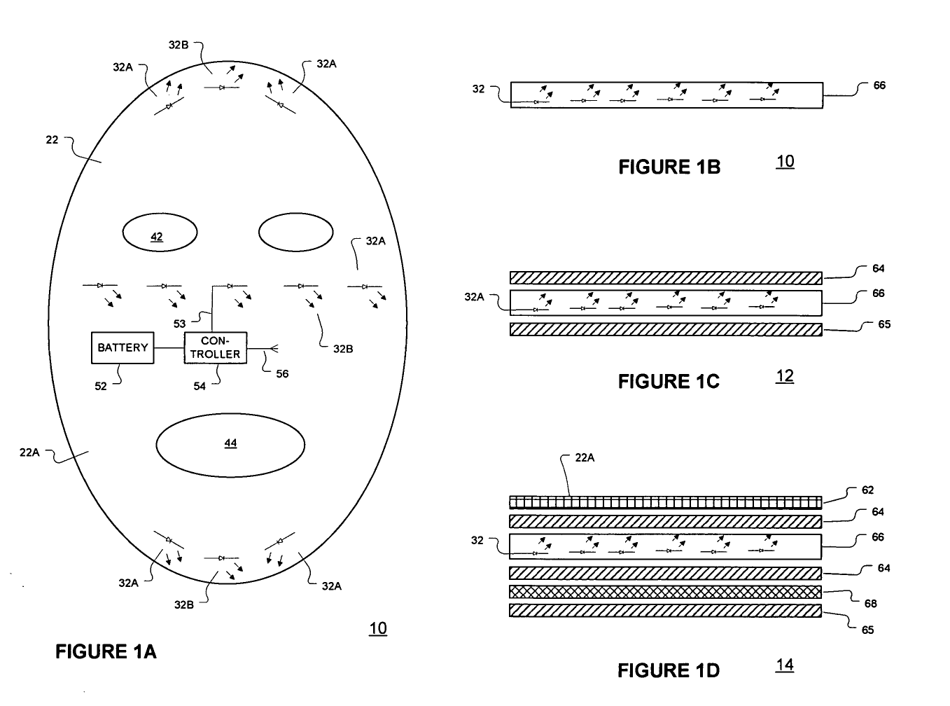

FIG. 1A is a simplified diagram of a somatic treatment system 10 according to various embodiments. The somatic treatment system 10 may have a surface 22 that includes a plurality of embedded LEDs 32A, 32B, a battery 52, a controller 54, and an antenna 56. In an embodiment the LED 32A may be configured to emit energy of a first particular frequency range and the LED 32B may be configured to emit energy of a second particular frequency range. The surface 22 may be embedded with a chemical 22A that may be used to treat somatic cells. The chemical 22A may be reactive to the first and the second frequency ranges. Further somatic cells may be reactive to the first and the second frequency ranges. In addition, the combination of the chemical 22A and the application of the first and the second frequency ranges to the chemical 22A and somatic cells may have a synergetic effect.

In an embodiment the chemical 22A may be applied directly to the somatic cells to be treated. In a further embodiment a chemical 22A may not be employed in addition to the system 10. The somatic treatment system 10 may include a single translucent layer 66 such as shown in FIG. 1B. The layer 66 may include one or more light emitting diodes (LED) 32A, 32B embedded in the layer, and a second translucent or opaque layer 65. The layer 66 may be comprised of a polyurethane, medical silicon, or other pliable, translucent, hypoallergenic material.

In another embodiment a somatic treatment system 12 may include a plurality of layers such as shown in FIG. 1C. The embodiment 12 may include a first translucent layer 64, the light emitting diode (LED) 66 layer, and a second translucent or opaque layer 65. The first translucent layer 64 may be comprised of a polyurethane, medical silicon, or other pliable, translucent, hypoallergenic material. The third layer 64 may also be comprised of a polyurethane, medical silicon, or other pliable, translucent or opaque, hypoallergenic material.

In another embodiment a somatic treatment system 14 may include a plurality of layers such as shown in FIG. 1D. The embodiment 14 may include a medicinal or chemical layer 62, a first translucent layer 64, a light emitting diode (LED) 66 layer, a second translucent layer 64, a reflective layer 68, and a second translucent or opaque layer 65. The first and second translucent layer 64 may be comprised a polyurethane, medical silicon, or other pliable, translucent, hypoallergenic material. The final layer 65 may also be comprised a polyurethane, medical silicon, or other pliable, translucent or opaque, hypoallergenic material. The medicinal or chemical layer 62 may include one or more chemicals 22A where the chemicals 22A may elude from the layer 62 due to interaction with somatic cells or first and second frequency ranges. The reflective layer may be comprised of any reflective material that reflect EMG energy towards the surface 22 including an aluminum foil material.

In an embodiment a local controller 54 and battery 52 may also be embedded in one the layers 66, 64, 65. The controller 54 may be electrically coupled to the one or more LEDS 32A, 32B. The controller 54 may also be coupled to a battery 52. The controller 54 may generate one or more signals for LEDs 32A, 32B as a function of a user switch 56. The signals may vary as a function of the first and second frequency ranges. The controller 54 may include one or more timers 58 that may limit the application of energy to the LEDs 32A, 32B to predetermined time intervals. In an embodiment the controller may also be coupled to an antenna 56 to receive or transmit one or more signals related to the transmission of energy to one or more LEDs 32A, 32B.

As shown in FIG. 1A the system 10 may be configured to treat a particular segment of somatic cells such as a face. The system 10 includes several openings 42, 44 for a user's eyes or mouth. The system 10 is configured to conform to a user's anatomy so that emitted light is focused on somatic cells. In another embodiment 200, the system 200 may be configured to drape about another anatomical region including somatic cells such as an arm, leg, chest, hands, feet, neck, or other region.

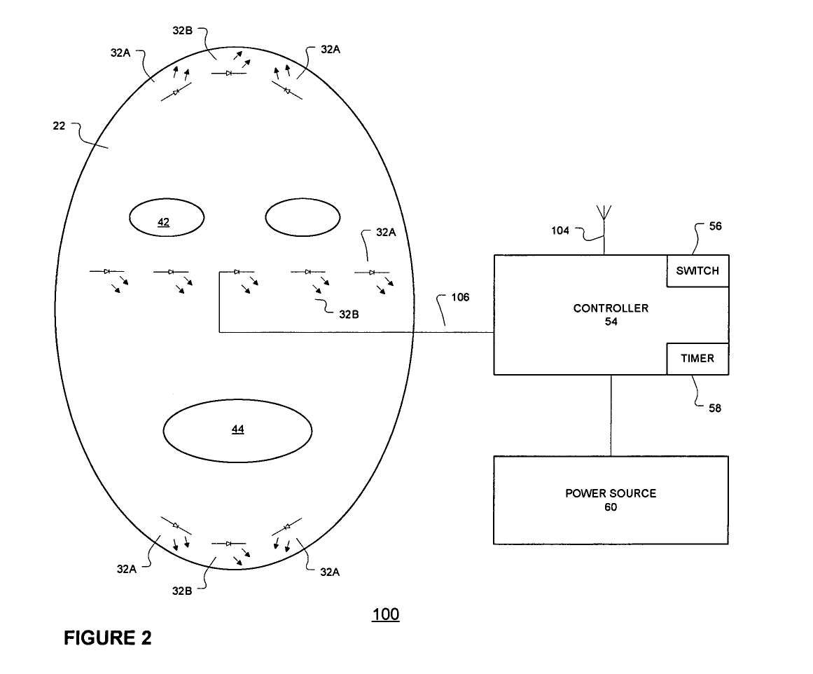

FIG. 2 is a simplified diagram of another somatic treatment system 100 according to various embodiments. The system 100 includes the LED transmission surface 22, a controller 54, an antenna 104, and a power source 60. In this embodiment the controller 54 may be located remote from the LED emission component 22. The power source 60 may be coupled to the controller 54. The controller 54 may be coupled to one or more LEDs 32A, 32B via one or more electric wires 106. The controller 54 may generate one or more signals for LEDs 32A, 32B as a function of the user switch 56. The signals may vary as a function of the first and second frequency ranges. The controller 54 may include one or more timers 58 that may limit the application of energy to the LEDs 32A, 32B to predetermined time intervals. In an embodiment the controller may also be coupled to an antenna 104 to receive or transmit one or more signals related to the transmission of energy to one or more LEDs 32A, 32B.

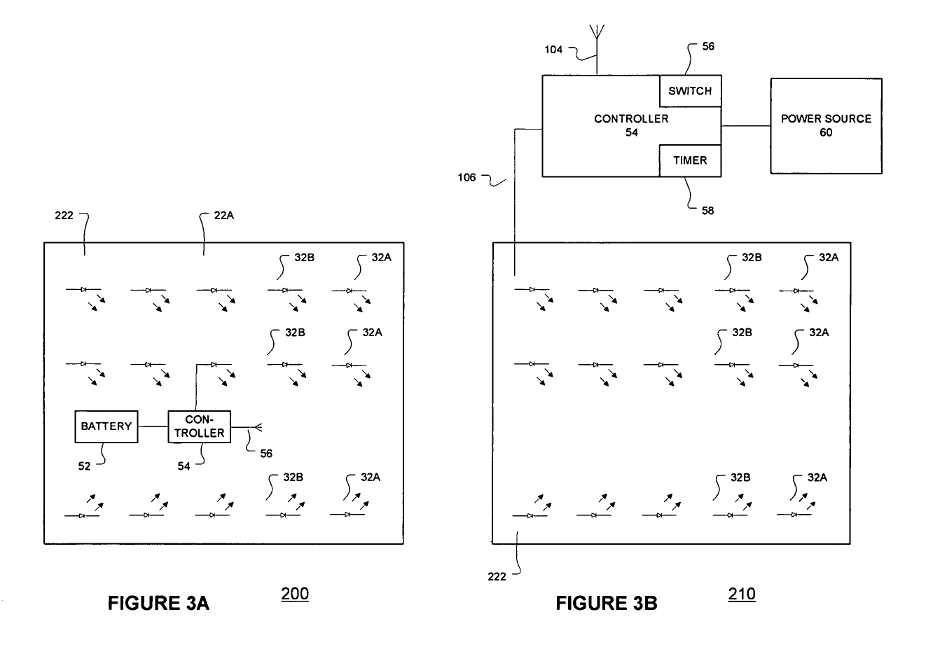

FIG. 3A is a simplified diagram of a somatic treatment system 200 according to various embodiments. The somatic treatment system 200 may have a surface 222 that includes a plurality of embedded LEDs 32A, 32B, a battery 52, a controller 54, and an antenna 56. In an embodiment the LED 32A may be configured to emit energy of a first particular frequency range and the LED 32B may be configured to emit energy of a second particular frequency range. The surface 22 may be embedded with a chemical 22A that may be used to treat somatic cells. The chemical 22A may be reactive to the first and the second frequency ranges. Further somatic cells may be reactive to the first and the second frequency ranges. In addition, the combination of the chemical 22A and the application of the first and the second frequency ranges to the chemical 22A and somatic cells may have a synergetic effect.

In an embodiment the chemical 22A may be applied directly to the somatic cells to be treated. In a further embodiment a chemical 22A may not be employed in addition to the system 200. The somatic treatment system 200 may include a single translucent layer 66 such as shown in FIG. 1B. The layer 66 may include one or more light emitting diodes (LED) 32A, 32B embedded in the layer, and a second translucent or opaque layer 65. The layer 66 may be comprised of a polyurethane, medical silicon, or other pliable, translucent, hypoallergenic material.

In another embodiment a somatic treatment system 12 may include a plurality of layers such as shown in FIG. 1C. The embodiment 12 may include a first translucent layer 64, the light emitting diode (LED) 66 layer, and a second translucent or opaque layer 65. The first translucent layer 64 may be comprised of a polyurethane, medical silicon, or other pliable, translucent, hypoallergenic material. The third layer 64 may also be comprised of a polyurethane, medical silicon, or other pliable, translucent or opaque, hypoallergenic material.

In another embodiment a somatic treatment system 14 may include a plurality of layers such as shown in FIG. 1D. The embodiment 14 may include a medicinal or chemical layer 62, a first translucent layer 64, a light emitting diode (LED) 66 layer, a second translucent layer 64, a reflective layer 68, and a second translucent or opaque layer 65. The first and second translucent layer 64 may be comprised a polyurethane, medical silicon, or other pliable, translucent, hypoallergenic material. The final layer 65 may also be comprised a polyurethane, medical silicon, or other pliable, translucent or opaque, hypoallergenic material. The medicinal or chemical layer 62 may include one or more chemicals 22A where the chemicals 22A may elude from the layer 62 due to interaction with somatic cells or first and second frequency ranges. The reflective layer may be comprised of any reflective material that reflects EMG energy towards the surface 222 including an aluminum foil material.

In an embodiment a local controller 54 and battery 52 may also be embedded in one the layers 66, 64, 65. The controller 54 may be electrically coupled to the one or more LEDS 32A, 32B. The controller 54 may also be coupled to a battery 52. The controller 54 may generate one or more signals for LEDs 32A, 32B as a function of a user switch 56. The signals may vary as a function of the first and second frequency ranges. The controller 54 may include one or more timers 58 that may limit the application of energy to the LEDs 32A, 32B to predetermined time intervals. In an embodiment the controller may also be coupled to an antenna 56 to receive or transmit one or more signals related to the transmission of energy to one or more LEDs 32A, 32B. The system 200 is configured to conform to a user's anatomy so that emitted light is focused on somatic cells. The system 200 may be configured to drape about another anatomical region including somatic cells such as an arm, leg, chest, hands, feet, neck, or other region.

FIG. 3B is a simplified diagram of another somatic treatment system 210 according to various embodiments. The system 210 includes the LED transmission surface 222, a controller 54, an antenna 104, and a power source 60. In this embodiment the controller 54 may be located remote from the LED emission component 22. The power source 60 may be coupled to the controller 54. The controller 54 may be coupled to one or more LEDs 32A, 32B via one or more electric wires 106. The controller 54 may generate one or more signals for LEDs 32A, 32B as a function of the user switch 56. The signals may vary as a function of the first and second frequency ranges. The controller 54 may include one or more timers 58 that may limit the application of energy to the LEDs 32A, 32B to predetermined time intervals. In an embodiment the controller may also be coupled to an antenna 104 to receive or transmit one or more signals related to the transmission of energy to one or more LEDs 32A, 32B.

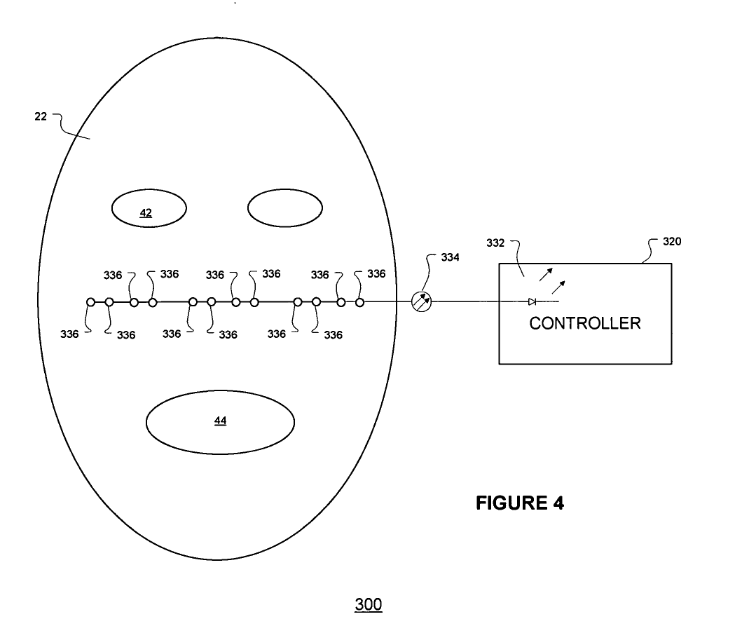

FIG. 4 is a simplified diagram of a somatic treatment system 300 according to various embodiments. In the system 300 the controller 320 may include LED lens 336, a fiber optic pathway 334, an LED 332. In this embodiment the LED 332 may be coupled to lens 336 via the fiber optic pathway 334. The controller 320 may generate an LED signal via the LED 332 that is transmitted to somatic cells via the lens 336 and the fiber optic pathway 334.

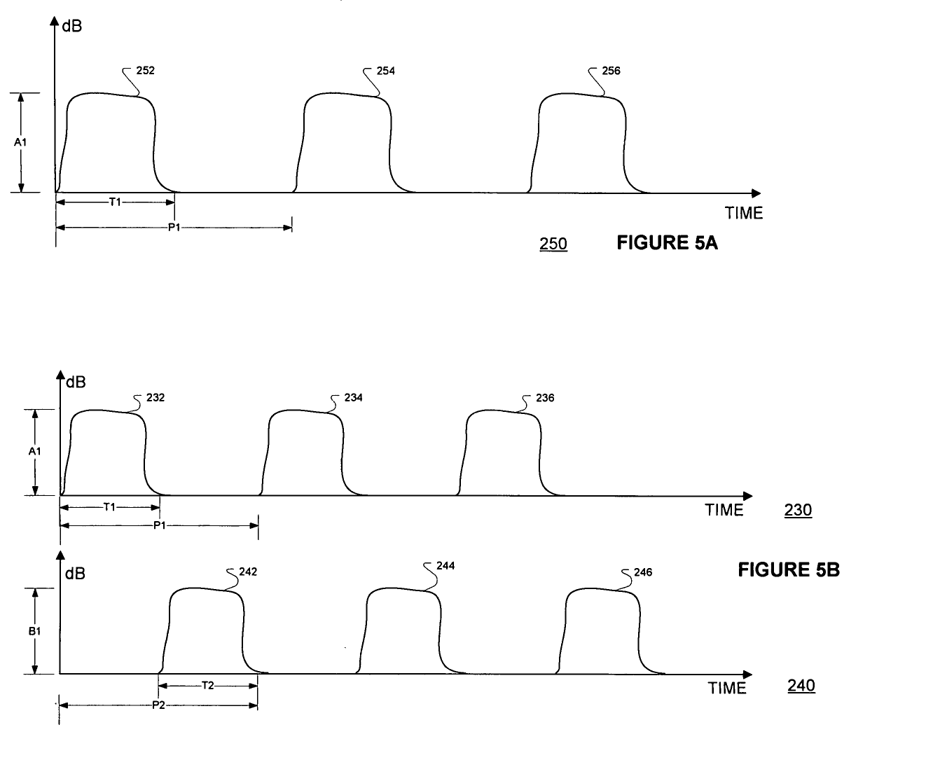

FIGS. 5A-5B are diagrams of electrical signal waveforms 230, 240, 250 that may be applied to one or more LEDs 32A, 32B, 332 according to various embodiments. The signal waveform 250 includes several square-wave pulses 252, 254, 256 that may be applied to an LED 32A, 32B, 332. Each pulse 252, 254, 256 may a have a similar magnitude and envelope. The waveform 250 may be used to energize an LED 32A, 32B, 332 periodically P1 for a predetermined interval T1 where each pulse 252, 254, 256 has a amplitude A1. In an embodiment, A1 may be about 0.1 milliamperes (mA) to 10 mA, the pulse width T1 may be about 100 microsecond (μs) to 500 μs and the period P1 may from 100 ms to 500 ms as a function of the energy required to create capacitance in a liquid. In another embodiment, A1 may be about 0.5 milliamperes (mA) to 5 mA, the pulse width T1 may be about 200 microsecond (μs) and the period P1 may about 250 ms as a function of the energy to create capacitance in a liquid.

In FIG. 5B a signal waveform 230 may be applied to a first LED 32A, 32B, 332 module or group and a second waveform 240 may be applied or used to energize a second LED 32A, 32B, 332 module. The signal waveform 230 includes several square-wave pulses 232, 234, and 236 and the signal waveform 240 includes several square-wave pulses 242, 244, and 246. Each pulse 232, 234, 236, 242, 244, 246 may a have a similar magnitude and envelope. The waveform 230 may be used to energize a first LED 32A, 32B, 332 module periodically P1 for a predetermined interval T1 where each pulse 232, 234, 236 has an amplitude A1. The waveform 240 may be used to energize a second LED 32A, 32B, 332 module periodically P2 for a predetermined interval T2 where each pulse 242, 244, 246 has an amplitude B1. The pulse width T1, T2 may be about 100 microsecond (μs) to 500 μs and the period P1, P2 may from 100 ms to 500 ms as a function of the energy to affect somatic cells or chemicals 22A. In another embodiment, A1, A2 may be about 0.5 milliamperes (mA) to 5 mA, the pulse width T1, T2 may be about 200 microsecond (μs) and the period P1, P2 may about 250 ms as a function of the energy required to affect somatic cells or chemicals 22A. In an embodiment the pulses 232, 234, 236 do not substantially overlap the pulses 242, 244, 246. In an embodiment T1>T2 and P2 is an integer multiple of P1.

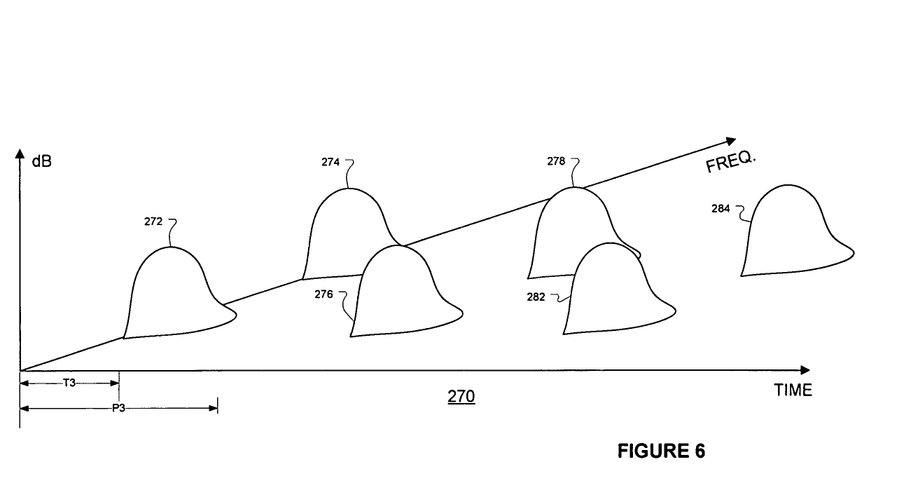

FIG. 6 depicts a waveform 270 that includes multiple pulses 272, 274, 276, 278, 282, and 284 that may not overlap in the time or the frequency domain. In an embodiment each pulse 272, 274, 276, 278, 282, and 284 may have a pulse width T3, and frequency spectrum width F1 and period P3. The pulse 272 is frequency offset from the pulse 274, the pulse 276 is frequency offset from the pulse 278, and the pulse 282 is frequency offset from the pulse 284. The pulses 272, 274, 276, 278, 282, and 284 may be applied to an LED module to affect somatic cells or chemicals 22A. Pulses 272, 274 having different frequency spectrums may enable different LED stimulation. In an embodiment the pulses 272, 276, 282 may be applied to a first LED module and the pulses 274, 278, 284 may be applied to a second LED module. The frequency separation between the respective pulses may enable simultaneous energization of a first and a second LED module and subsequent and independent spectrum generation.

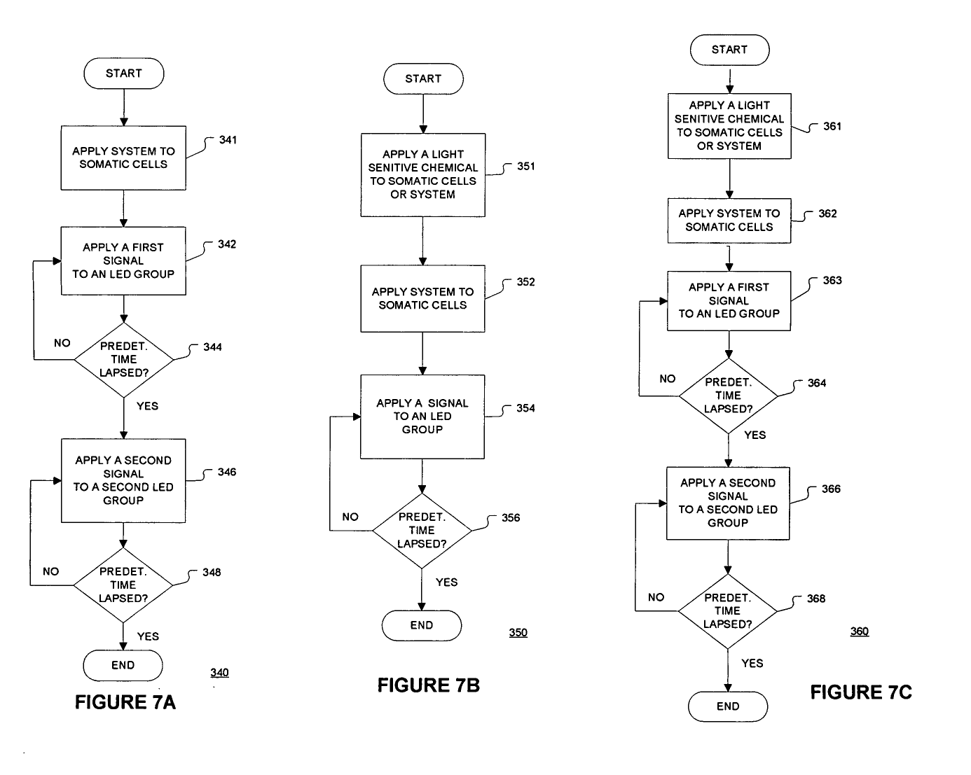

In an embodiment the invention may employ the algorithm 340 shown in FIG. 7A to apply therapy to somatic cells. A user, clinician, or equipment may place a system 10, 100, 200, 210 on somatic cells to be treated (activity 341). A first signal such as shown in FIGS. 5A, 5B, and 6 may be applied to a first LED module or group (32A) of a somatic system 10, 100, 200, 210 (activity 342) for a predetermined time period (activity 344). A second signal such as shown in FIGS. 5A, 5B, and 6 may be applied to a second LED module or group (32B) of a somatic system 10, 100, 200, 210 (activity 346) for a predetermined time period (activity 348). The signals applied to the groups may be selected to stimulate somatic cells or chemicals 22A.

In another embodiment the invention may employ the algorithm 350 shown in FIG. 7B to apply therapy to somatic cells. A user, clinician, or equipment may apply a light sensitive chemical on a system 10, 100, 200, 210 or on somatic cells to be treated (activity 351). The user, clinician, or equipment may place a system 10, 100, 200, 210 on somatic cells to be treated (activity 352). A signal such as shown in FIGS. 5A, 5B, and 6 may be applied to a LED module or group (32A or 32B) of a somatic system 10, 100, 200, 210 (activity 354) for a predetermined time period (activity 356).

In another embodiment the invention may employ the algorithm 360 shown in FIG. 7C to apply therapy to somatic cells. A user, clinician, or equipment may apply a light sensitive chemical on a system 10, 100, 200, 210 or on somatic cells to be treated (activity 361). The user, clinician, or equipment may place a system 10, 100, 200, 210 on somatic cells to be treated (activity 362). A first signal such as shown in FIGS. 5A, 5B, and 6 may be applied to a first LED module or group (32A) of a somatic system 10, 100, 200, 210 (activity 363) for a predetermined time period (activity 364). A second signal such as shown in FIGS. 5A, 5B, and 6 may be applied to a second LED module or group (32B) of a somatic system 10, 100, 200, 210 (activity 366) for a predetermined time period (activity 368).

The systems 10, 100, 200, 210, 310 may be used to employ cosmetic or medications or other chemicals directly on somatic cells such as skin with the addition of light of specific frequencies for treatment and healing of epidermal cells of the skin or tissue below the skin with the object of assisting the agents used in delivery, uptake, action and function more effectively. The LEDs 32A, 32B may create the specific frequencies of light. The system 10, 100, 200, 210, 310, light application may enable cosmetic or medication or other active chemicals 22A on somatic cells for longer time periods while preventing dehydration of the applied substances. Such light application may improve the efficacy of cosmetic or medication or other active chemical as a function of the selected wavelengths or frequencies.

Further the somatic system application may increase cellular activity and help heal tissue faster and facilitate the delivery, uptake and use in the cell of the cosmetics, medications, or chemicals 22A used. The LED light of specific frequencies can increase fibroblast production and collagen as well as other activities of the cell including stimulating the organells and mitochondria to produce ATP for cell energy for functioning, decreasing treatment time and facilitate healing. The system 10, 100, 200, 210, 300 make the agents used on the body more efficacious and useful to the body on a cellular level.

In the present invention Light-emitting diodes (LED's) 32A, 32B may produce multiple wavelengths, and may be arranged in large, flat arrays allowing treatment of large wounds or areas of the body or somatic regions such as shown in FIGS. 1A, 2, 4. A system 10, 100, 200, 210, 300 may be employed to treat serious burns, crush injuries, non-healing fractures, muscle and bone atrophy, enhancing muscle growth, traumatic ischemic wounds, radiation tissue damage, compromised skin grafts, cancerous tissue, wrinkles, telangiectasia and cellulite, dark spots on skin, muscle aches, sun burn, lymphadenitis, superficial phlebitis, inflamed varicose veins, various inflammatory process, e.g. furuncles and carbuncles, reduction of bruises and scarring, softening of the skin, reduction or elimination of inflammation, unwanted tattoos of natural or intentional origin, and tissue regeneration including skin and hair.

The systems 10, 100, 200, 210, 300 may stimulate the basic energy processes in the mitochondria (energy compartments) of each cell, particularly when near-infrared light is used to activate the color sensitive chemicals (chromophores, cytochrome systems) inside but not limited to these spectrum alone as the UV, other visible and IR spectrums may also be usable. In an embodiment optimal LED wavelengths for skin repair may include 640, 680, 730 nanometers (nm) wavelengths to IR 880 nm. Further application of blue light 400 nm to 490 via the system 10, 100, 200, 210, 300 may inhibit the growth and kill bacteria, fungus in and on somatic cells.

The system 10, 100, 200, 210, 300 may be employed to apply cosmetics, medications and/or other actives directly to the skin and maintain their presence long-term while using LED or other actinic light to increase their effect on the cells and tissue in the body. Due to the flexibility of the embodiments, LED light or other actinic lighting may be shone onto body surfaces that are not flat while providing even light distribution to the desired treatment area. The systems 10, 200 are also highly portability and enable user mobility during treatment.

It is noted that the systems include flexible sheets that may contain cosmetics, medications or other chemicals that may cover a somatic body part and generated LED or other actinic light that may reacts with somatic cells and the active ingredients with or without a photoinitiator to increase absorption and efficacy of the cosmetic, medication or other chemicals 22A to decrease the healing time for the selected target somatic cells or decrease the time needed for other desirable effects such as smoothing surface texture, reducing acne, or other clinical effects.

The system 10, 100, 200, 210, 300 may include a semi permanent flexible body masking for a somatic area to stop dehydration and maintain the proper dosage or amount of cosmetic, medication or other chemicals to affect the cells of the body while allowing exposure of the selected somatic cells to actinic light from LED or other actinic light source. It is noted that the chemical 22A may include a photo reactive substance such as a silver ion, PDT drugs, beta-carotenes, pigments or any other photo reactive substance that may increase energy states of molecules and/or an increase in the efficacy and reaction of the cells of the body when using LED or other light of certain wavelengths activate the substance that is kept on the body via the system 10, 100, 200, 210, 300 containing one or more LED's 32A, 32B or other light source. The system 10, 100, 200, 210, 300 may be used with a trans-dermal delivery substance, e.g., the Latitude Pharmaceutical Dermal Delivery System.

The system 10, 100, 200, 210, 300 may also reduce the appearance of cellulite, body shaping; decrease wrinkles (rhytides), aid in acne reduction, aid in skin rejuvenation or reduce the appearance of vascular lesions, decrease the effects of age, sun damage, spider veins, vitiligo, psoriasis, and treat AK's, cancer, and pigmented skin of the face or body. The system may enable the delivery of agents and lights for the use in Photodynamic Therapy (PDT). The system may also be employed to selectively mark (illuminate) and kill target cells while covering the skin of the body by shading them from the procedure light and ambient or sun light during and following the PDT procedure.

The system 10, 100, 200, 210, 300 may also be employed to selectively remove hair from the body permanently with or without photoinitiators, medication and light or grow hair (alopecia) in a selected area with the use of medical, herbal or other agents and using light for added benefit and effect a device that is more universally functional in today's market than the prior art devices. The system 10, 100, 200, 210, 300 may also be placemd inside the oral cavity, rectum, vaginal canal and uterus, inside the ear and even surgically placed internally within the body. It will also be understood that, in addition to the human use where medication and light can be used in healing, the device can be used to treat other primates, horses, cats, dogs, cattle or other livestock, pets or animals in zoos, or in the treating animals in the wild.

It is noted that layers may include a laminate which includes a moisture bearing layer which is placed against somatic cells. Both the moisture bearing layer and the outer sheet of relatively liquid impermeable material are clear or translucent so that the therapy light can penetrate to the tissues being treated. It is noted that the array of LED light sources 32A, 32B may be part of a fine net of connecting wires (53 in FIG. 1A) that may be attached to a small power source 52, 60 such as a flat disk battery via a controller 54. The LED 32A, 32B array may be embedded into either a gel layer or a clear flexible material such as silicone 66. In an embodiment the layer 66 may include a hydrogel or other moist material.

It is noted that two or more systems 10, 100, 200, 210, 300 may be attached to each other with a strap, a Velcro patch or other locking or interlocking mechanism to add additional segments or detach segments. It is noted that the chemical 22A may include a photoinitiator or other photoreactive substance. The layers 62, 64, 66, 68 may be a flexible sheet made of clear or translucent polypropylene, silicone, or other clear plastic such as polyurethane film or PVC, or hydrocolloid or gel, sized to the particular part of the body it will be used to cover. It is flexible and able to conform to the body. It could be of a size to surround a leg, arm, shoulder, foot, hand, head, nose, eye and/or face (although not limited to those parts alone but any body feature or prominence), and flexible enough for the wearer to sleep, walk or move with the device attached to the body. A layer may be held fast to the skin using a dermal ‘holding’ and delivery system or adhesive material.

Chemicals 22A may include cosmetics, medications and other actives appropriate for somatic cells including AHA's (alpha hydroxy acid), natural oils, aloe vera compounds, collagen boosters, bt, chitosan, daeses, endorphins, photodynamic drugs (PDT) like (Photofrin or ALA), vitamins A, C E or others, kojic acid, retinols or other exfoliant, salicylic acid, anti oxidants or other youth boosters and anti aging cosmetic or medications, antiseptic, antibiotics, anti-cancer agents, aroma therapy agents, fruit and vegetable extracts, anti-inflammatory agents, pain relievers, hormones, depilatories, and others, but the scope of this invention is not limited to these alone but can include any helpful medication, herbal formula or active compound for the skin and/or other tissues.

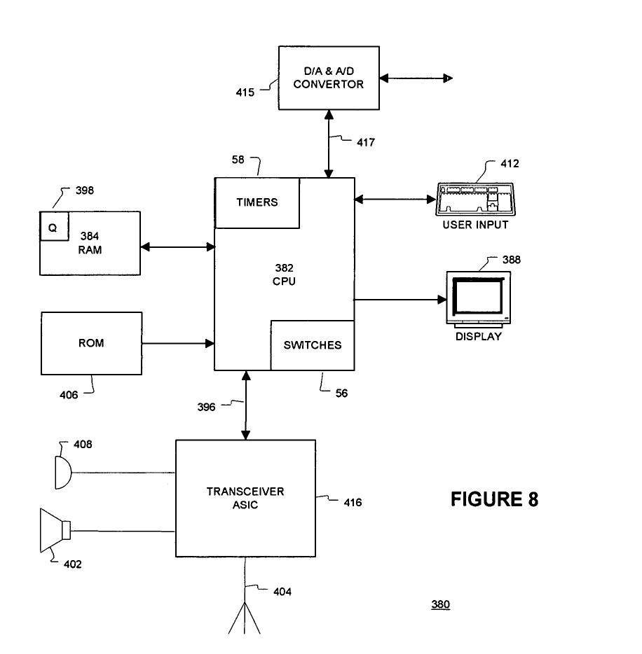

FIG. 8 is a block diagram of an article 380 according to various embodiments. The article 380 shown in FIG. 10 may be used in various embodiments as a part of a system 10, 100, 200, 210, 300 where the article 380 may be any computing device including a personal data assistant, cellular telephone, laptop computer, or desktop computer. The article 380 may include a central processing unit (CPU) 382, a random access memory (RAM) 384, a read only memory (ROM″) 406, a display 388, a user input device 412, a transceiver application specific integrated circuit (ASIC) 416, a digital to analog (D/A) and analog to digital (A/D) convertor 415, a microphone 408, a speaker 402, and an antenna 404. The CPU 382 may include an OS module 414 and an application module 413. The RAM 384 may include switches 56 and timers 58.

The ROM 406 is coupled to the CPU 382 and may store the program instructions to be executed by the CPU 382. The RAM 384 is coupled to the CPU 382 and may store temporary program data, overhead information, and the queues 398. The user input device 412 may comprise an input device such as a keypad, touch pad screen, track ball or other similar input device that allows the user to navigate through menus in order to operate the article 380. The display 388 may be an output device such as a CRT, LCD, LED or other lighting apparatus that enables the user to read, view, or hear user detectable signals.

The microphone 408 and speaker 402 may be incorporated into the device 380. The microphone 408 and speaker 402 may also be separated from the device 380. Received data may be transmitted to the CPU 382 via a bus 396 where the data may include signals for an LED 32A, 32B, 332 or optical module. The transceiver ASIC 416 may include an instruction set necessary to communicate data, screens, or signals. The ASIC 416 may be coupled to the antenna 404 to communicate wireless messages, pages, and signal information within the signal. When a message is received by the transceiver ASIC 416, its corresponding data may be transferred to the CPU 382 via the serial bus 396. The data can include wireless protocol, overhead information, and data to be processed by the device 380 in accordance with the methods described herein.

The D/A and A/D convertor 415 may be coupled to one or more optical modules to generate a signal to be used to energize one of the optical modules. The D/A and A/D convertor 415 may also be coupled to one devices such as LEDs 32A, 32B. Any of the components previously described can be implemented in a number of ways, including embodiments in software. Any of the components previously described can be implemented in a number of ways, including embodiments in software. Thus, the LEDs 32A, 32B, controllers 54, switch 56, timers 58, controller 320 may all be characterized as “modules” herein. The modules may include hardware circuitry, single or multi-processor circuits, memory circuits, software program modules and objects, firmware, and combinations thereof, as desired by the architect of the system 10, 30, 50, 60 and as appropriate for particular implementations of various embodiments.

The apparatus and systems of various embodiments may be useful in applications other than a sales architecture configuration. They are not intended to serve as a complete description of all the elements and features of apparatus and systems that might make use of the structures described herein.

Applications that may include the novel apparatus and systems of various embodiments include electronic circuitry used in high-speed computers, communication and signal processing circuitry, modems, single or multi-processor modules, single or multiple embedded processors, data switches, and application-specific modules, including multilayer, multi-chip modules. Such apparatus and systems may further be included as sub-components within a variety of electronic systems, such as televisions, cellular telephones, personal computers (e.g., laptop computers, desktop computers, handheld computers, tablet computers, etc.), workstations, radios, video players, audio players (e.g., mp3 players), vehicles, medical devices (e.g., heart monitor, blood pressure monitor, etc.) and others. Some embodiments may include a number of methods.

It may be possible to execute the activities described herein in an order other than the order described. Various activities described with respect to the methods identified herein can be executed in repetitive, serial, or parallel fashion.

A software program may be launched from a computer-readable medium in a computer-based system to execute functions defined in the software program. Various programming languages may be employed to create software programs designed to implement and perform the methods disclosed herein. The programs may be structured in an object-orientated format using an object-oriented language such as Java or C++. Alternatively, the programs may be structured in a procedure-orientated format using a procedural language, such as assembly or C. The software components may communicate using a number of mechanisms well known to those skilled in the art, such as application program interfaces or inter-process communication techniques, including remote procedure calls. The teachings of various embodiments are not limited to any particular programming language or environment.

The accompanying drawings that form a part hereof show, by way of illustration and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In the foregoing Detailed Description, various features are grouped together in a single embodiment for the purpose of streamlining the disclosure. This method of disclosure is not to be interpreted to require more features than are expressly recited in each claim. Rather, inventive subject matter may be found in less than all features of a single disclosed embodiment. Thus the following claims are hereby incorporated into the Detailed Description, with each claim standing on its own as a separate embodiment.

What is claimed is:

1. A method of treating somatic tissue, comprising:
placing a flexible translucent material including at least one embedded light generation device therein on or near somatic tissue to be treated; and
energizing the light generation device.

2. The method of claim 1, further comprising placing a chemical on at least a portion of the somatic tissue and at least a portion of the translucent material inner surface.

3. The method of claim 1, further comprising placing a chemical on a portion of the translucent material inner surface.

4. The method of claim 2, wherein the chemical is a photoactive chemical.

5. The method of claim 1, comprising placing a flexible translucent material including at least one embedded LED therein on or near somatic tissue to be treated; and energizing the LED.

6. The method of claim 1, comprising placing a flexible translucent material including at least one embedded infra-red LED therein on or near somatic tissue to be treated; and energizing the LED to generate an infra-red signal toward somatic tissue to be treated.

7. The method of claim 1, comprising placing a flexible translucent material including at least one embedded infra-red LED therein on or near somatic tissue to be treated; and energizing the LED to generate an infra-red light signal toward somatic tissue to be treated for a predetermined time interval.

8. The method of claim 1, comprising placing a flexible translucent material including at least one embedded LED, a power source, and a controller coupling the LED to the battery therein on or near somatic tissue to be treated; and energizing the LED to generate an infra-red signal toward somatic tissue to be treated for a predetermined time interval.

9. The method of claim 1, comprising placing a reflective layer on an outer surface of the flexible translucent material, the outer surface opposite the inner surface.

10. The method of claim 1, comprising placing a flexible translucent material including at least two embedded LEDs therein on or near somatic tissue to be treated; and energizing the two LED to generate light energy toward somatic tissue to be treated for a predetermined time interval.

11. A apparatus for treating somatic tissue, comprising:
a flexible translucent material;
a light generation module embedded in the flexible translucent material; and
an energizing module for energizing the light generation module.

12. The apparatus for claim 11, further comprising a chemical on at least a portion of the translucent material inner surface.

13. The apparatus for claim 11, further comprising a controller module embedded in the translucent material, the controller module controlling the energization of the light generation module.

14. The apparatus for claim 12, wherein the chemical is a photoactive chemical.

15. The apparatus for claim 11, wherein the light generation module is a LED module.

16. The apparatus for claim 11, wherein the light generation module is an infra-red LED module.

17. The apparatus for claim 16, further comprising a controller module embedded in the translucent material, the controller module controlling the energization of the infra-red module for a predetermined time interval.

18. The apparatus for claim 17, further comprising an embedded power source, the power source coupled to the controller module.

19. The apparatus for claim 11, further comprising a reflective layer on an outer surface of the flexible translucent material, the outer surface opposite the inner surface.

20. The apparatus for claim 11, wherein the LED modules includes a plurality of LEDs.

21. An article of manufacture for use in treating somatic tissue, the article of manufacture comprising computer readable storage media including program logic embedded therein that causes control circuitry to perform energizing a light generation device embedded in a flexible translucent material, the flexible translucent material located therein on or near somatic tissue to be treated.
